(19) United States
(12) Patent Application Publication
(10) Pub. No.: US 2010/0204146 A1
(43) Pub. Date: Aug. 12, 2010

(54) Treatment of Oestrogen Dependant Conditions in Pre-menopausal Women

(75) Inventor: Ernest LOUMAYE, Plan-les-Ouates/Geneva (CH)

Correspondence Address: STERNE, KESSLER, GOLDSTEIN & FOX P.L.L.C., 1100 NEW YORK AVENUE, N.W., WASHINGTON, DC, 20005, US

(73) Assignee: PregLem S.A., Plan-Les-Ouates/Geneva (CH)

(21) Appl. No.: 12/678,674

(22) PCT Filed: Sep. 4, 2008
(86) PCT No.: PCT/IB2008/002296

§ 371 Date: Mar. 17, 2010

Related U.S. Application Data
(60) Provisional application No. 60/960,109, filed on Sep. 17, 2007.

(51) Int. Cl. A61K 38/08; A61K 31/56; A61P 15/00

(52) U.S. Cl. 514/15; 514/182; 514/170; 514/171

(57) ABSTRACT

The present invention relates to a method for treating and/or preventing ovarian cycle disturbance, prolonged un-opposed secretion of estrogens, and/or ovarian follicular cyst formation in pre-menopausal women with functional ovaries when treated with steroid sulfatase inhibitors (STS-I) for an estrogen-dependant condition.

### FIELD OF THE INVENTION

The present invention relates to a method for treating and/or preventing ovarian cycle disturbance, prolonged un-opposed secretion of estrogens, and/or ovarian follicular cyst formation in pre-menopausal women with functional ovaries when treated with a steroid sulfatase inhibitors (STS-I) for an estrogen-dependant condition.

### BACKGROUND OF THE INVENTION

Several severe conditions occurring in pre-menopausal women are estrogen-dependant. These include benign conditions such as endometriosis, adenomyosis, uterus myomas, and benign breast fibro-cystic dysplasia as well as malignant conditions such as breast cancer, endometrium cancer, ovarian cancer.

Endometriosis is characterized by the presence of endometrium-like tissue outside the uterus cavity, most frequently in the peritoneal cavity. Endometrium almost exclusively affects pre-menopausal women. Endometriosis is a highly prevalent and highly under-diagnosed condition. There are an estimated 7 million endometriosis patients in the U.S., 12-14 million endometriosis patients in Europe and estimated 80 million in the Rest of World. Endometriosis is a major cause of chronic pelvic pain, dyspareunia and sub-fertility. Proliferation and growth of endometrial tissue is estrogen-dependant.

Treatments for endometriosis currently aim at suppressing menstruation and oestrogen production by the ovary. This is achieved by danazol and progestins or GnRH agonists. These products alleviate pain symptoms in half of the patients. However, these products use is limited to 6 months for GnRH agonists because of potential adverse effect on bone mineral density and treatment with danazol is also limited because of its androgenic side-effects. Moreover, symptoms recurrence is reported in a majority of the patients within 5 years of treatment cessation. Thus, there remain significant unmet needs for better long term therapies. STS activity has been detected in normal (eutopic) and hyperplastic endometrium, as well as ectopic endometrium i.e. pelvic endometriosis. Moreover, in vitro, STS-I (i.e. STX64) has been shown to abolish the sulfatase activity present in eutopic and ectopic endometrium (A. Purohit et al., 2005; World Congress of Endometriosis, Maastricht & Annual Meeting Endocrine Society, San Diego, 2005). These authors also reported that the STS activity is much more consistently and quantitatively observed in endometriosis than CYP19.

STS expression has also been demonstrated in another form of endometriosis i.e. adenomyosis (K. Ezaki et al., Obstetrics and Gynecology 2001; 98: 815-819).

Therefore, the Applicants have evaluated the therapeutic relevance of inhibiting the STS activity in pre-menopausal women with endometriosis.

Breast cancer is a highly prevalent condition which concerns both pre- and post-menopausal women. It is a major health problem affecting as many as one in eight women during their lifetime. With 1 million new cases in the world each year, breast cancer is the commonest malignancy in women and comprises 18% of all female cancers. It is the cause of death in over 400,000 women annually.

Overall the incidence of breast cancer rises with age, increasing rapidly during the fourth decade of life and continuing to increase thereafter, but more slowly in the fifth, sixth and seventh decades.

In the USA, 25% of new diagnoses of breast cancer are in women below the age of 50 (most being pre-menopausal women) and 75% of new diagnoses of breast cancer are in women aged 50 years or older (most being post-menopausal women).

Treatment include surgery, radiation therapy, chemotherapy and hormonal therapy.

A majority of breast cancer expresses estrogen receptor and tumor growth is estrogen dependant. Hence the use of estrogen suppressing agent such as aromatase inhibitor is the mainstay of breast cancer adjuvant therapy.

It is well known that Steroid sulfatase enzyme (STS) expression is increased in breast cancer tumors and sulfatase activity is significantly higher than the aromatase activity in post-menopausal breast cancer patients (J R Pasqualini et al. J Clin Endocrinol Metab 81:1460-1464, 1996). STS expression in the tumour has prognostic significance. STS mRNA expression is significantly associated with poor prognosis only in estrogen-receptor positive tumors (Y. Miyoshi et al. Clin Cancer Res, 9: 2288-2293, 2003). The role of STS in supporting tumor growth prompted the development of potent STS inhibitors. Initial successful testing of a potent irreversible sulfatase inhibitor in patients with advanced stage breast cancer has been recently reported (S. J. Stanway et al., Clin Cancer Res 2006; 12 (5)). Therefore, the Applicants have evaluated the therapeutic relevance of inhibiting the STS activity in pre-menopausal women with breast cancer.

Surprisingly, they have found that administration of steroid sulfatase inhibitor to adult female non-human primates and pre-menopausal women disturb ovulation and the menstrual cycle resulting in delayed or absence of ovulation and delayed or absence of menstruation. Additionally, ovarian cyst formation was observed.

These adverse outcomes are unpleasant and can reduce the quality of life, and generate anxiety for the patients. They can also potentially results in more significant complications such as persisting ovarian cysts which may be associated with pain and torsion. In addition, this is associated with fluctuating and persistent estrogen levels which can partially counteract the therapeutic benefit of STS-I, by exposing the estrogen dependent tissue to high levels of circulating oestrogens.

Therefore, there was a need for improving the safety and the efficacy of steroid sulfatase inhibitors (STS-I) treatment in pre-menopausal women with functional ovaries when treated with such STS-I for a benign or a malignant, estrogen-dependant conditions.

This object has been achieved by co-administering in pre-menopausal women with functional ovaries and treated with a steroid sulfatase inhibitor (STS-I), a therapeutically effective amount of a compound selected from the group comprising a progesterone agonist (progestin), an oral combined estrogen and progestin contraceptive pill and/or a GnRH analog.

### SUMMARY OF THE INVENTION

The present invention concerns a method for treating and/or preventing ovarian cycle disturbance, prolonged un-opposed secretion of estrogens, and/or ovarian follicular cyst formation in pre-menopausal women with functional ovaries when treated with a steroid sulfatase inhibitors (STS-I), or an active metabolite thereof, for an estrogen-dependant condition.

A further object of the present invention is to provide a pharmaceutical composition comprising a combination of a steroid sulfatase inhibitor (STS-I), or an active metabolite thereof, with a therapeutically effective amount of a compound, or an active metabolite of said compound, selected from the group comprising a progesterone agonist (progestin), an oral combined estrogen and progestin contraceptive pill and/or a GnRH analog.

Still another object of the invention is to provide methods of treatment of endometriosis, breast cancer, uterus myoma, and breast benign fibro-cystic dysplasia.

### DETAILED DESCRIPTION OF THE INVENTION

The present invention concerns a method for treating and/or preventing ovarian cycle disturbance, prolonged un-opposed secretion of estrogens, and/or ovarian follicular cyst formation in pre-menopausal women with functional ovaries when treated with a steroid sulfatase inhibitors (STS-I), or an active metabolite thereof, for an estrogen-dependant condition comprising co-administering a therapeutically effective amount of a compound, or an active metabolite of said compound, selected from the group comprising a progesterone agonist (progestin), an oral combined estrogen and progestin contraceptive pill (OC pill) and/or an GnRH analog.

As used herein, the following definitions are supplied in order to facilitate the understanding of the present invention.

“Ovarian or menstrual cycle disturbance”: Human menstrual cycle results from a precisely timed sequence of events in the ovaries comprising follicular growth, ovulation and luteal phase. In healthy women, the whole process takes on average 28 days (from the first day of menstruation to the day before next menstruation). Individual women usually have a minimal variation in terms of duration (number of days) of their menstrual cycle. The average duration of the human menstrual cycle is 28 days but it may vary between 21 and 35 days. The ovarian or menstrual cycle disturbance is a modification of the individual menstrual cycle duration by either expending or shortening the cycle duration.

“Prolonged un-opposed secretion of estrogens”: During the follicular phase of the menstrual cycle, the growing follicle is secreting increasing amount of estrogens. Following ovulation (at mid-cycle), the estrogen secretion decreases and progesterone is massively secreted by the ovary. Progesterone counteracts many of the estrogen effects such as estrogen-induced cell proliferation. Disruption of the ovulation mechanism leads to prolonged estrogen secretion which is not followed by a proper phase of progesterone secretion. Prolonged un-opposed secretion of estrogen is a secretion of estrogen beyond the normal duration of 14 days, without appropriate sequential secretion of progesterone.

“Ovarian follicular cyst formation”: The ovarian follicle is a structure which contains the oocyte (ovum). The center of the structure is filled with a fluid (follicular fluid). Ovulation is the rupture of the follicle into the abdominal cavity and the release of the oocyte. If no ovulation occurs, fluid accumulates in the follicle which growths well beyond the normal size at pre-ovulatory stage (i.e. diameter 20-25 mm). The enlarged follicle is a cyst which size may exceed 10 cm in diameter.

“Pre-menopausal women”: A pre-menopausal women is a woman with ovaries which are capable to ovulate. It extends from puberty to menopause.

“Functional ovaries”: Ovaries are functional when ovulating. Typically, ovaries are functional between puberty and menopause.

The terms “treating” or “treatment” both refer to therapeutic treatment and prophylactic or preventative measures. Those subjects in need of treatment include those already with the disorder as well as those in which the disorder is to be prevented. Hence, the subject to be treated herein may have been diagnosed as having the disorder or may be predisposed or susceptible to the disorder.

The term “estrogen” as used herein includes natural estrogens such as estrone, estrone sulfate, estrone sulfate piperazine salt, estradiol and estriol, and their esters, as well as ethinyl estradiol, mestranol, conjugated equine estrogen, esterified estrogens, estropipate, 17[alpha]-ethinylestradiol, esters and ethers of 17[alpha]-ethinylestradiol such as, for example, 17[alpha]-ethinylestradiol 3-dimethylamino propionate, 17[alpha]-ethinylestradiol 3-cyclopentyl ether (quinestrol) and 17[alpha]-ethinylestradiol 3-methyl ether (mestranol), estradiol-17beta, estradiol valerate, piperazine estrone sulphate, estriol succinate, and polyestrol phosphate and other estrogen equivalents and estrogen agonists and antagonists.

“Estrogen-dependant condition” refers to a disease, a condition, a tumor which initiation, and/or proliferation and/or growth is stimulated by estrogens.

“Estrogen-dependant condition is a benign condition or a malignant condition”: Conditions which are estrogen-dependant can either be benign (local proliferation without metastasis) or malignant (local proliferation associated with metastasis). A malignant condition is typically a cancer.

“An oral combined estrogen and progestin contraceptive pill”: orally active contraception aims at suppressing ovulation to prevent conception in women with functional ovaries. This is achieved by the combined administration of a synthetic estrogen and a synthetic progestin.

The term “comprise” is generally used in the sense of include, that is to say permitting the presence of one or more features or components.

As used herein, “a” or “an” means “at least one” or “one or more.”

The term “SPRM” stands for selective progesterone receptor modulator and represents a class of progesterone receptor ligands that exerts clinically relevant tissue-selective progesterone agonist, antagonist, or partial (mixed) agonist/antagonist effects on various progesterone target tissues in an in-vivo situation depending on the biological action studied (Smith C L and O'Malley B W, 2004, Coregulator function: a key to understanding tissue specificity of selective receptor modulators in Endocr Rev 25:45-71.)

An “active metabolite”, as used herein, is a product produced through metabolism in the body of a specified compound or salt thereof and which exhibits the same biological activity as the specified compound.

Active metabolites may be identified using routine techniques known in the art and their activities determined using tests. Such metabolites may result for example from the oxidation, reduction, hydrolysis, amidation, deamidation, esterification, deesterification, enzymatic cleavage, and the like, of the administered STS-I or compound selected from the group comprising a progesterone agonist (progestin), an oral combined estrogen and progestin contraceptive pill and/or a GnRH analog. Accordingly, the invention includes active metabolites of STS-I or of a compound selected from the group comprising a progesterone agonist (progestin), an oral combined estrogen and progestin contraceptive pill and/or a GnRH analog, including compounds produced by a process comprising contacting a compound of this invention with a mammal for a period of time sufficient to yield a metabolic product thereof. Such metabolite may also be produced in vitro by oxidation, reduction, hydrolysis, amidation, deamidation, esterification, deesterification, or enzymatic cleavage of the corresponding STS-I or of the compound selected from the group comprising a progesterone agonist (progestin), an oral combined estrogen and progestin contraceptive pill and/or a GnRH analog.

Examples of active metabolites of progesterone antagonists or SPRM are shown in the following table 1:

| Progesterone antagonist/SPRM | Active Metabolite |
|---|---|
| asoprisnil | J912 |
| | J956 |
| CDB-4124 | CDB-4453 |
| CDB-2914 | CBD-3877, |
| | CDB-3963, |
| | CDB-3236 and |
| | CDB-4183 |

This includes (but is not limited to) in the present invention compounds such as the steroid sulfatase inhibitor E1 MATE (estrone sulfamate) which is an ‘active metabolite’ of the steroid sulfatase inhibitor E2MATE (estradiol sulfamate) or SPRM monodemethylated CDB2914 which an ‘active metabolite’ of the SPRM CDB2914 or SPRM CDB-4453 (monodemethylated CDB4124) an ‘active metabolite’ of the SPRM CDB4124.

“Co-administering”, as it applies in the present invention, refers to contact of at least two pharmaceutical agents or compositions, to the subject, preferably a human, simultaneously, separately or concomitantly.

A “therapeutically effective amount” is an amount effective to ameliorate, treat or prevent the symptoms, diseases or disorders in a subject.

As used herein, “a progesterone receptor agonist” or “a progesterone agonist” refers to a compound or agent that activates the activity of the progesterone receptor. “Progesterone agonist” and “progestin” are used interchangeably herein.

Surprisingly, Applicants have found that co-administration of a progestin, an oral combined estrogen and progestagen contraceptive pill or a GnRH analog, or an active metabolite of these compounds, allows preventing cycle irregularity, cyst formation and irregular estrogen secretion hence enhancing the safety and the efficacy of STS-I in pre-menopausal women with functional ovaries treated with said STS-I.

In the present invention a sulfatase inhibitor (STS-I) is defined as a compound that prevents active estrogens to be formed from their biologically inactive sulfated forms, and active androgens to be formed from their biologically inactive sulfated forms by inhibiting the steroid sulfatase enzyme.

Steroid sulfatase enzyme (STS) is responsible for the hydrolysis of aryl and alkyl steroid sulfates and therefore has a pivotal role in regulating the formation of biologically active steroids. STS is responsible for the hydrolysis of estrone sulfate and dehydroepiandrosterone sulfate to estrone and dehydroepiandrosterone respectively. Estrone is biologically active as an estrogen and can be further converted in estradiol, the most potent oestrogen, and DHEA can be converted to steroids with estrogenic activity. (M J Reed Endocrine Reviews 26: 171-202, 2005).

STS activity has been detected in many tissues including breast, most tissues of the female reproductive tract and the skin. High STS activity has been found in endometriosis tissue and breast cancer tissue.
Sulfated oestrogens and androgens are very abundant and STS enzyme converts them into active forms.
Examples of Steroid Sulfatase inhibitors (STS-I) include but are not limited to:
EMATES such as E1 MATE or E2MATE as described in WO93/05063 (Sterix Limited) and WO93/05064 (Sterix Limited)

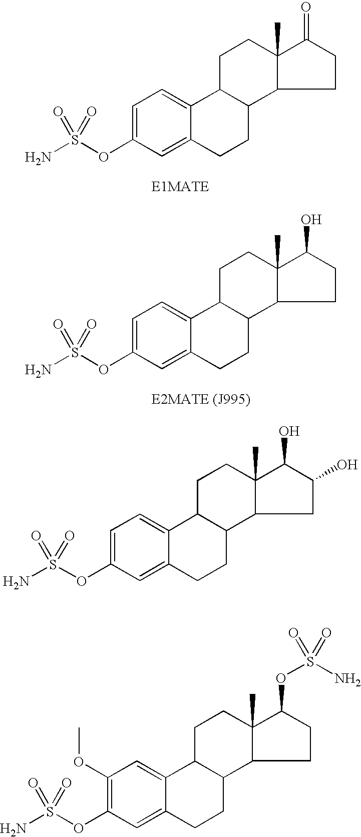

ANGIOMATES such as compound 1 described in WO98/24802 (Sterix Limited) and WO00/066095 (Sterix Limited)

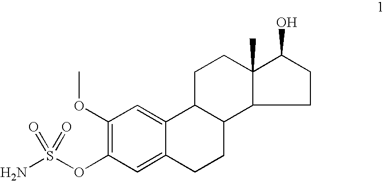

UREAMATES such as compound 2 claimed in WO03/033518 (Sterix Limited) and WO00/232409 (Sterix Limited).

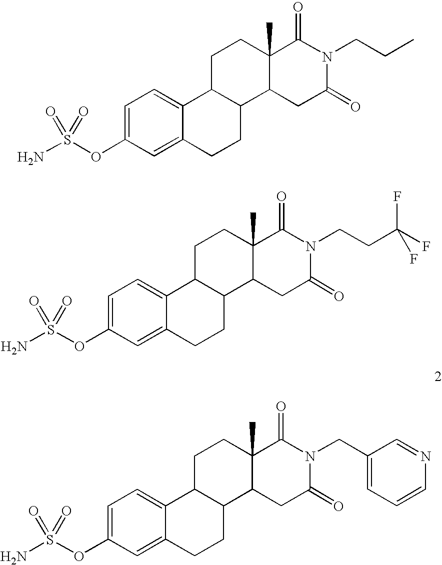

COUMATES such as 667COUMATE (STX-64) as described in WO97/30041 (Sterix Limited)

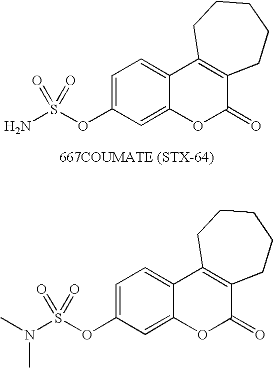

D-RING SULFAMATES such as compound 3 described in WO02/16392 (Sterix Limited)

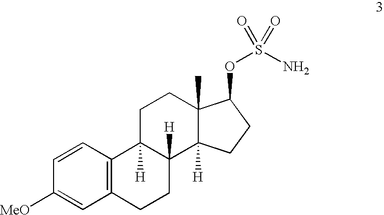

CHROMANOMATE such as compound of 0.4 as described in WO99/52890 (Novartis AG)

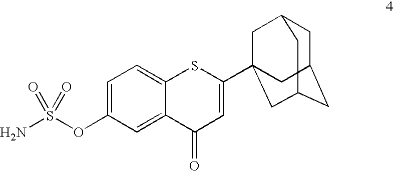

BENZAZOLMATE such as compound 5 as described in WO001/36398 (Novartis AG)

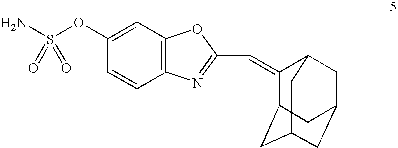

NORTROPINYLSULFONYUREA such as compound 6 described in WO2006/097292 (Novartis AG)

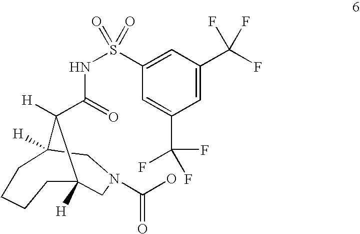

PIPERIDINESULFONYLUREA of formula (I) with preferably R1=Br, Cl, F (mono or disubstituted), CF3 and R2=COOtBu or aryl as described in WO03/082842 (Novartis AG)

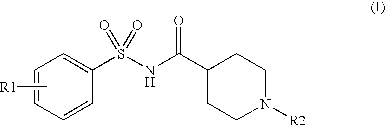

THIAZOLOSULFONYLUREA of formula (II) with preferably R1=R2=Aryl as described in WO04/043968 (Novartis AG)

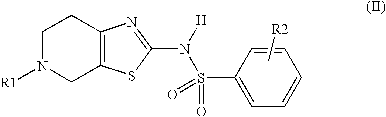

BRIDGED PIPERIDINESULFONYLUREA formula (III) with preferably R1=Br, Cl, F (mono or disubstituted), CF3 and R2=COOtBu or aryl amide as described in WO03/031397 (Novartis AG).

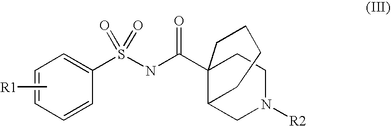

Dual Sulfatase and Aromatase inhibitors (DASI) such as compounds 7 and 8 as described in WO03/045925 (Sterix Limited), WO05/118560 (Sterix Limited) and WO05/115996 (Sterix Limited) and WO05/058842 (LABORATOIRE THERAMEX).

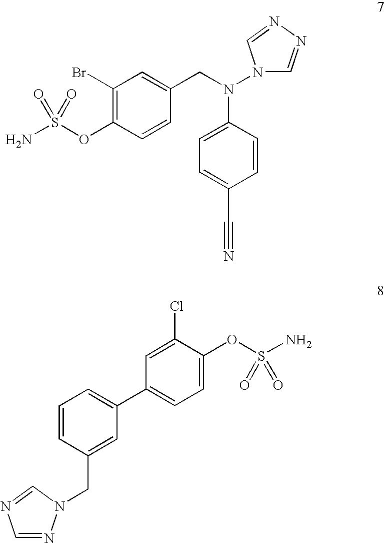

BIPHENYLSULFAMATE such as compound 9 described in WO07068905 (Sterix Limited)

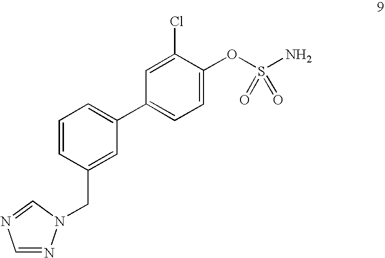

All the above cited references are incorporated in the present description/invention by reference.

The administration dose of STS-I, or of an active metabolite thereof, is variable and will depend essentially of the compound used, its pharmacokinetic and pharmacodynamic characteristics, as well as its mode of administration. Usually, the STS-I is to be administered at a dose between 0.25 mg-10 mg weekly oral (dose compares with the range of 0.2 mg/kg-2.0/kg in monkeys as described in example 1).

As used herein, a “progestin” is defined as a natural or synthetic progestational substance that mimics some or all of the actions of progesterone. Examples of progestins include but are not limited to derivatives of 19-nortestosterone, such as oestranes, and gonanes, and derivatives of 17 α-cetoxyprogesterone (pregnanes). Examples of oestranes include: norethindrone and its acetate, and ethynodiol diacetate. Examples of gonanes include norgestrel and levonorgestrel and the less androgenic derivatives of levonorgestrel such as desogestrel, norgestimate, and gestodene. Drospirenone is an other example of synthetic progestin.

The dose of progestin, or of an active metabolite thereof, is variable and will depend essentially of the compound used, its pharmacokinetic and pharmacodynamic characteristics, as well as its mode of administration. Usually, the progestin is to be administered at a dose between 0.05 to 0.20 mg/day.
Preferably, the progestin is levonorgestrel which is administered at a dose between 0.05 to 0.15 mg/day, preferably 0.1 mg/day.
A particular class of progestins is the selective progesterone receptor modulators which display partial agonist and antagonist properties. The SPRM that are consider for this invention are selected from the group comprising, but not limited to, CDB2914, mifepristone, asoprisnil, proellex, onapristone, org33628, tanproget, tanaproget-combo, WAY 166989, NSP 989, NSP-Combo, and 11 [beta]-benzaldoxime substituted SPRMs or an active metabolite of these SPRM.
The dose of SPRM, or of an active metabolite thereof, is variable and will depend essentially of the compound used as well as its mode of administration. Usually, the SPRM is to be administered at a dose between 2.5 to 20 mg/day. Preferably, the SPRM is CDB2914 to administer at a dose of 2.5 to 20 mg/day, preferably 10 mg/day.

The oral combined estrogen and progestin contraceptive pill (OC pill) which are commonly used include tablets comprising: (i) ethinyl estradiol and norethindrone; (ii) ethinyl estradiol and norgestimate; (iii) ethinyl estradiol and desogestrel; (iv) ethinyl estradiol and levonorgestrel; (v) ethinyl estradiol and gestodene; (vi) ethinyl estradiol and norgestrel; (vii) mestranol and norethindrone.

Preferably, the combination pill is ethinyl estradiol and levonorgestrel at a dose of 0.015 to 0.100 mg/day, preferably 0.020 mg/day for ethinyl estradiol and between 0.05 and 0.15 mg/day, preferably 0.1 mg/day for levonorgestrel.

In the present invention the term “GnRH” refers to Gonadotrophin releasing hormone which is a peptidic hormone secreted by a specific area of the brain called hypothalamus. This decapeptide plays a pivotal role in the mechanisms of reproduction in many species and specifically in humans.

A “GnRH analog” is defined as a natural (or native) or synthetic analog, either agonist or antagonist of GnRH receptor. A natural GnRH can be obtained from different sources such as i) natural sources (e.g. from urine, hypothalamus, placenta or gonads), ii) chemical synthesis (e.g peptide synthesis), iii) or recombinant techniques, where the GnRH amino acid sequence is encoded by a cloned gene and expressed and recovered from expression cells. In the two latter cases (ii and iii) the GnRH shares the same amino acid sequence as the GnRH obtained from natural sources.

The GnRH analog may also derive from native GnRH analog. In such case, this non-native (or non-natural) GnRH analog comprises an amino acid sequence derived from native GnRH which is different from the amino acid sequence of the native GnRH.
Analogs derived from native GnRH structure have been synthezised and selected for an agonist activity that is enhanced compared to the native peptide. This increased activity is mainly due to an enhanced resistance to degradation and a higher affinity for the pituitary GnRH receptor (Loumaye E et al., 1982, Binding affinity and biological activity of gonadotropin releasing hormone agonists in isolated pituitary cells. Endocrinology; 111:730-736).
Examples of GnRH analog agonist derived from native GnRH include but are not limited to buserelin, triptorelin, nafarelin, leuprolide, historelin, goserelin and a like Preferblay, these GnRH analgos are in the form of slow-release (SRF) or immediate release form (IRF) of buserelin, triptorelin, nafarelin, leuprolide, historelin, goserelin and a like. A preferred GnRH analog agonist derived from native GnRH is leuprolide, more preferably a SRF of leuprolide.

The term “GnRH antagonist” refers to synthetic or natural analogs of the native GnRH or analogs derived from native GnRH which have the capacity to recognize and inactivate or block GnRH receptors. Examples of GnRH analog antagonists include but are not limited to SRF and IRF forms of cetrorelix, ganirelix, degarelix, teverelix, abarelix and alike. A preferred GnRH antagonist is a SRF of degarelix.

The administration frequency of the STS-I and the compound selected from the group comprising a progesterone agonist (progestin), an oral combined estrogen and progestin contraceptive pill (OC pill) and/or an GnRH analog is also critical and must be defined for each STS-I and co-administered compound in regards of their pharmacokinetic and pharmacodynamic properties as well as their formulation. Usually, the administration is carried continuously for at least 3 to 6 months. However, treatment for 1 or 2 years is also envisaged. Treatment for greater than 2 years is contemplated.

It is envisioned that co-administering the therapeutically effective amount of the compound is started before the STS-I start. Usually, the administration of the therapeutically effective amount of the compound is started concomitantly with the STS-I administration. Alternatively, the administration of the therapeutically effective amount of the compound is started following or sequentially, with or without overlapping, with the STS-I administration.

Also encompassed in the present invention is a pharmaceutical composition comprising a combination of an STS-I, or an active metabolite thereof, with a therapeutically effective amount of a compound selected from the group comprising a progesterone agonist (progestin), an oral combined estrogen and progestin contraceptive pill and/or a GnRH analog, or active metabolites thereof. This pharmaceutical composition may be formulated as follows: (i) the STS-I and the progestin mixed together in a single formulation; (ii) the STS-I and the OC pill mixed together in a single formulation; (iii) the STS-I and the GnRH analog mixed together in a single formulation; or (iv) each component formulated separately, for simultaneous or sequential dosing.

The pharmaceutical composition comprising a combination of an STS-I with a therapeutically effective amount of a compound selected from the group comprising a progesterone agonist (progestin), an oral combined estrogen and progestin contraceptive pill and/or a GnRH analog for use in the method as described herein, is usually in the form of a pharmaceutical composition that may contain one or more pharmaceutically acceptable carriers, such as excipients, carriers and/or auxiliaries which facilitate processing of the active compounds into preparation which can be used pharmaceutically.
Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS® or polyethylene glycol (PEG).

The form of administration of the pharmaceutical composition may be systemic or topical. For example, administration of such a composition may be various entreral or parenteral routes such as oral, vaginal, rectal, subcutaneous, intravenous, intradermal, intramuscular, intraperitoneal, intranasal, transdermal, buccal routes or via an implanted device, and may also be delivered by peristaltic means.

One variation of the present invention also foresees a pharmaceutical composition suitable for delayed and controlled release of the Progesterone agonist and SPRM of the pharmaceutical composition as defined in the present invention. The Progesterone agonist and SPRM, for example, may be incorporated in a matrix of biocompatible polymer allowing delayed and controlled release. All biocompatible polymers, well known by those skilled in the art are potential candidate to be used in this invention.

Sustained-release preparations may be prepared. Suitable examples of sustained-release preparations include semi permeable matrices of solid hydrophobic polymers containing the progesterone agonist or SPRM, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma]ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. The formulations to be used for in vivo administration must be sterile. This is readily accomplished for example by filtration through sterile filtration membranes.

It is understood that the suitable dosage of the pharmaceutical composition of the present invention will be dependent upon the age, health, and weight of the woman in need thereof, kind of concurrent treatment, if any and the nature of the effect desired. The suitable dosage form will also depend on the disease, the pharmaceutical composition, and the mode of administration; possibilities include tablets such as pills, capsules, lozenges, dental pastes, suppositories, inhalants, solutions, ointments and parenteral depots.

Alternatively, or additionally, it will become apparent that the pharmaceutical composition may be administered alone or in combination with other treatments, therapeutics or agents, either simultaneously or sequentially dependent upon the condition to be treated.

Additionally, the present invention also envisioned a kit comprising

i) a pharmaceutical composition comprising a combination of a steroid sulfatase inhibitors (STS-I) with a therapeutically effective amount of a compound selected from the group comprising a progesterone agonist (progestin), an oral combined estrogen and progestin contraceptive pill and/or an GnRH analog,
ii) and optionally with reagents and/or instructions for use.
Also embraced in the scope of this invention are the following methods of treatment:

of endometriosis comprising the administration of a STS-I (0.25 mg-8 mg, weekly oral administration) and a progestin (0.1 mg-10 mg, daily oral administration) including a SPRM,

of endometriosis comprising the administration of a STS-I (0.25 mg-8 mg, weekly oral administration) and an oral combined estrogen (ethinylestradiol, 10 μg-100 μg daily oral administration) and progestin contraceptive pill (0.1 mg-10 mg daily oral administration),

of endometriosis comprising the administration of a STS-I (0.25 mg-8 mg, weekly oral administration) and a GnRH agonist (immediate release form: 500-1500 μg daily; slow release form: 1 mg-5 mg monthly

of endometriosis comprising the administration of a STS-I (0.25 mg-8 mg, weekly oral administration) and a GnRH antagonist (0.1 mg-5 mg daily subcutaneous or intramuscular),

of breast cancer comprising the administration of a STS-I (0.25 mg-8 mg, weekly oral administration) and a progestin (0.1 mg-10 mg, daily oral administration) including a SPRM,

of breast cancer comprising the administration of a STS-I (0.25 mg-8 mg, weekly oral administration) and an oral combined estrogen and progestin contraceptive pill (0.1 mg-10 mg, daily oral administration),

of breast cancer comprising the administration of a STS-I (0.25 mg-8 mg, weekly oral administration) and a GnRH agonist,

of breast cancer comprising the administration of a STS-I (0.25 mg-8 mg, weekly oral administration) and a GnRH antagonist,

of uterus myoma comprising the administration of a STS-I (0.25 mg-8 mg, weekly oral administration) and a progestin including a SPRM,

of uterus myoma comprising the administration of a STS-I (0.25 mg-8 mg, weekly oral administration) and an oral combined estrogen and progestin contraceptive pill,

of uterus myoma comprising the administration of a STS-I (0.25 mg-8 mg, weekly oral administration) and a GnRH agonist,

of uterus myoma comprising the administration of a STS-I (0.25 mg-8 mg, weekly oral administration) and a GnRH antagonist,

of breast benign fibro-cystic dysplasia comprising the administration of a STS-I (0.25 mg-8 mg, weekly oral administration) and a progestin including a SPRM,

of breast benign fibro-cystic dysplasia comprising the administration of a STS-I (0.25 mg-8 mg, weekly oral administration) and an oral combined estrogen and progestin contraceptive pill,

of breast benign fibro-cystic dysplasia comprising the administration of a STS-I (0.25 mg-8 mg, weekly oral administration) and a GnRH agonist,

of breast benign fibro-cystic dysplasia comprising the administration of a STS-I (0.25 mg-8 mg, weekly oral administration) and a GnRH antagonist,

Also envisioned in the present invention is the use a pharmaceutical composition comprising a combination of a steroid sulfatase inhibitors (STS-I) with a therapeutically effective amount of a compound selected from the group comprising a progesterone agonist (progestin), an oral combined estrogen and progestin contraceptive pill and/or an GnRH analog for the treatment and/or prevention of ovarian cycle disturbance, prolonged un-opposed secretion of estrogens, and/or ovarian follicular cyst formation in pre-menopausal women with functional ovaries when treated with a steroid sulfatase inhibitors (STS-I) for an estrogen-dependant condition.

Another object of the invention is the use a pharmaceutical composition comprising a combination of a steroid sulfatase inhibitors (STS-I) with a therapeutically effective amount of a compound selected from the group comprising a progesterone agonist (progestin), an oral combined estrogen and progestin contraceptive pill and/or an GnRH analog for the treatment of endometriosis, breast cancer, uterus myoma, and breast benign fibro-cystic dysplasia.

The foregoing description will be more fully understood with reference to the following Examples. Such Examples, are, however, exemplary of methods of practicing the present invention and are not intended to limit the scope of the invention.

### EXAMPLES

### Example 1

Estradiol sulfamate (E2MATE) is a potent steroid sulfatase inhibitor. Three groups of eight female Cynomolgus monkeys (Macaca fascicularis) each were treated intragastrically over a period of approximately 39 weeks at daily doses of 0.2, 0.8 and 2.0 mg/kg of E2MATE using an application volume of 10 mL/kg. A fourth group (control) received an equivalent volume of the vehicle under the same experimental conditions.
The impact on ovarian cycle and function were as follows:
From 0.2 mg/kg/day onwards, the frequency and incidence of blood stained vaginal discharge was decreased and associated with a prolongation of the menstrual cycle up to complete amenorrhea (i.e. absence of menstruation).
At the low dose of 0.2 mg/kg, a compound-related prolongation of the menstrual cycle length was observed in 6/8 animals while 2/8 animals became amenorrheic during the treatment period.
For the intermediate and the highest dose of 0.8 mg/kg and 2.0 mg/kg respectively, a persistent amenorrhea in most animals dominated the menstrual cycle pattern during the whole treatment period.
After 39 weeks of treatment, the microscopic status at necropsy was as follows:

| | Dose group | | | |
|---|---|---|---|---|
| | 0 mg/kg | 0.2 mg/kg | 0.8 mg/kg | 4.0 mg/kg |
| No. animals | 8 | 8 | 8 | 8 |
| Ovary: Absence of | 0 | 7 | 8 | 8 |
| corpora lutea* | (0%) | (87.5%) | (100%) | (100%) |
| Ovary: Increase | 0 | 4 | 1 | 4 |
| in growing follicles | (0%) | (50%) | (12.5%) | (50%) |
| Ovary: Cyst | 2 | 1 | 1 | 0 |
| | (25%) | (12.5%) | (12.5%) | (0%) |

* Corpus luteum (corpora lutea) is the histological evidence for ovulation.

This study in non-human primate clearly documents that administration of E2MATE, a potent steroid sulfates inhibitor, disturbs ovulation, induces anovulation and amenorrhea. This is associated in a significant proportion of subjects with multiple follicular growths and sometimes with ovarian cyst formation.

### Example 2

A 35 year-old healthy woman with regular, ovulatory menstrual cycle is exposed to a STS-I for assessing the pharmacokinetic of the compound from day 2 of her menstrual cycle onwards. The STS-I is a steroidal arylsulfamate compound administered at a dose of 1 mg/week. The follicular development is monitored with frequent serum estradiol measurements, and trans-vaginal ultrasound of the ovaries. Her serum estradiol profile during the follicular phase displays abnormally slow increase in estradiol levels, while several follicles growth simultaneously. At day 14 of her cycle, the trans-vaginal ultrasound shows the presence of several large follicles (diameter ranging between 14 and 25 mm) and an unexpectedly low serum estradiol level for the extend of follicle development. On day 21 of the cycle, follicles are still present but larger (range between 20 and 40 mm) and estradiol remains elevated. No significant progesterone serum levels are measured. All together this indicates absence of ovulation. On day 28 she does not menstruate, estradiol remain elevated. On day 35, she starts having some spotting and one cyst is present in each ovary.
After induction of menstruation by administration of a progestin for 7 days, the subject started an OC pill in association with the arylsulfamate compound. The cysts spontaneously resolved. During the co-administration, no follicular development is recorded and serum estradiol remain low (around 50 pg/ml). Menstrual bleeding occurs at regular, monthly interval.

### Example 3

A 32 year old, nulliparous female was diagnosed with a moderate/severe peritoneal and ovarian endometriosis during a laparoscopy performed for chronic pelvic pain, dyspareunia and infertility. Despite careful debulking of the lesions during the laparoscopy, the symptoms persisted and medical treatment is indicated. A STS-I, E2MATE is administered at 1.0 mg/week. Symptoms were partially relieved, but irregular episodes of mild but painful vaginal bleeding was reported. Serum estradiol measurements indicated fluctuating serum E2 in the hundred of pg range. A progestin, norgestrel (0.1 mg/day) is associated with E2MATE leading to a reduction of serum estradiol levels and of bleeding episode, with further improvement of the patient's symptoms.

### Example 4

In a 42 year-old women, a metastatic breast cancer is progressing despite administration of aromatase inhibitor and a GnRH agonist. At the time of surgery, the tumour has been shown to be positive for estrogen receptor and STS expression. STX64, a STS-I from the coumate family of compound is administered on a daily basis. The GnRH agonist is maintained, no follicular development is recorded and her serum estradiol levels remain low (20 to 30 pg/ml).

1. A method for treating and/or preventing ovarian cycle disturbance, prolonged un-opposed secretion of estrogens, and/or ovarian follicular cyst formation in pre-menopausal women with functional ovaries when treated with a steroid sulfatase inhibitors (STS-I), or an active metabolite thereof, for an estrogen-dependant condition comprising co-administering a therapeutically effective amount of a compound, or an active metabolite of said compound, selected from the group comprising a progesterone agonist (progestin), an oral combined estrogen and progestin contraceptive pill and/or a GnRH analog.

2. The method of claim 2 wherein the GnRH analog is native or non native.

3. The method of claim 1 or 2 wherein the GnRH analog is an agonist or an antagonist of the GnRH.

4. The method of claims 1 to 2 wherein the compound is co-administered separately or concomitantly.

5. The method of claims 1 to 4 wherein the estrogen-dependant condition is a benign condition or a malignant condition.

6. The method of claims 1-5, wherein the STS-I is selected from the group comprising EMATES, ANGIOMATES, UREAMATES, COUMATES, D-RING SULFAMATES, CHROMANOMATE, BENZAZOLMATE, NORTROPINYLSULFONYUREA, PIPERIDINESULFONYLUREA, THIAZOLOSULFONYLUREA, BRIDGED PIPERIDINESULFONYLUREA, Dual Sulfatase and Aromatase inhibitors (DASI) and BIPHENYLSULFAMATE, or an active metabolite thereof.

7. The method of claims 1-5, wherein the progestin is selected from the group comprising derivatives of 19-nortestosterone derivatives of 17 α-acetoxyprogesterone (pregnanes), levonorgestrel, drospirenone, and selective progesterone receptor modulators (SPRM).

8. The method of claim 6, wherein the derivatives of 19-nortestosterone are selected from the group comprising oestranes, and gonanes.

9. The method of claim 7, wherein the oestranes are selected from the group comprising norethindrone and its acetate, and ethynodiol diacetate.

10. The method of claim 7, wherein the gonanes are selected from the group comprising norgestrel and levonorgestrel and the less androgenic derivatives of levonorgestrel (desogestrel, norgestimate, and gestodene).

11. The method of claim 6, wherein the selective progesterone receptor modulators (SPRM) are selected from the group comprising CDB2914, mifepristone, asoprisnil, proellex, onapristone, org33628, tanproget, tanaproget-combo, WAY 166989, NSP 989, NSP-Combo, and 11[beta]-benzaldoxime substituted SPRMs.

12. The method of claims 1-10, wherein the OC pill is selected from the group comprising (i) ethinyl estradiol and norethindrone; (ii) ethinyl estradiol and norgestimate; (iii) ethinyl estradiol and desogestrel; (iv) ethinyl estradiol and levonorgestrel; (v) ethinyl estradiol and gestodene; (vi) ethinyl estradiol and norgestrel; (vii) mestranol and norethindrone.

13. The method of claims 1-11, wherein the GnRH agonist is selected from the group comprising slow-release (SRF) and immediate release form (IRF) of buserelin, triptorelin, nafarelin, leuprolide, historelin, goserelin and a like.

14. The method of claims 1-11, wherein the GnRH antagonist is selected from the group comprising slow-release (SRF) and immediate release form (IRF) of cetrorelix, ganirelix, degarelix, teverelix, abarelix and alike.

15. The method of claims 1-13, wherein co-administering the therapeutically effective amount of the compound is started before the STS-I start.

16. The method of claims 1-13, wherein administering the therapeutically effective amount of the compound is started concomitantly with the STS-I administration.

17. The method of claims 1-13, wherein administering the therapeutically effective amount of the compound is started following or sequentially, with or without overlapping, with the STS-I administration.

18. A pharmaceutical composition comprising a combination of a steroid sulfatase inhibitors (STS-I) with a therapeutically effective amount of a compound selected from the group comprising a progesterone agonist (progestin), an oral combined estrogen and progestin contraceptive pill and/or an GnRH analog.

19. The pharmaceutical composition of claim 17 wherein the GnRH analog is an agonist or an antagonist of the GnRH.

20. A method of treatment of endometriosis comprising the administration of a STS-I and a progestin including a SPRM

21. A method of treatment of endometriosis comprising the administration of a STS-I and an oral combined estrogen and progestin contraceptive pill.

22. A method of treatment of endometriosis comprising the administration of a STS-I and a GnRH agonist.

23. A method of treatment of endometriosis comprising the administration of a STS-I and a GnRH antagonist.

24. A method of treatment of breast cancer comprising the administration of a STS-I and a progestin including a SPRM.

25. A method of treatment of breast cancer comprising the administration of a STS-I and an oral combined estrogen and progestin contraceptive pill.

26. A method of treatment of breast cancer comprising the administration of a STS-I and a GnRH agonist.

27. A method of treatment of breast cancer comprising the administration of a STS-I and a GnRH antagonist.

28. A method of treatment of uterus myoma comprising the administration of a STS-I and a progestin including a SPRM

29. A method of treatment of uterus myoma comprising the administration of a STS-I and an oral combined estrogen and progestin contraceptive pill.

30. A method of treatment of uterus myoma comprising the administration of a STS-I and a GnRH agonist.

31. A method of treatment of uterus myoma comprising the administration of a STS-I and a GnRH antagonist

32. A method of treatment of breast benign fibro-cystic dysplasia comprising the administration of a STS-I and a progestin including a SPRM.

33. A method of treatment of breast benign fibro-cystic dysplasia comprising the administration of a STS-I and an oral combined estrogen and progestin contraceptive pill.

34. A method of treatment of breast benign fibro-cystic dysplasia comprising the administration of a STS-I and a GnRH agonist.

35. A method of treatment of breast benign fibro-cystic dysplasia comprising the administration of a STS-I and a GnRH antagonist.

36. A kit comprising
i) a pharmaceutical composition comprising a combination of a steroid sulfatase inhibitors (STS-I)), or an active metabolite thereof, with a therapeutically effective amount of a compound), or an active metabolite of said compound, selected from the group comprising a progesterone agonist (progestin), an oral combined estrogen and progestin contraceptive pill and/or an GnRH analog,
ii) and optionally with reagents and/or instructions for use.
